(19) United States
(12) Patent Application Publication
(10) Pub. No.: US 2026/0221248 A1
(43) Pub. Date: Jul. 30, 2026

(54) MEDICAL INFORMATION GENERATION DEVICE AND MEDICAL INFORMATION GENERATION METHOD

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 19/351,336

(22) Filed: Oct. 7, 2025

(30) Foreign Application Priority Data
Jan. 27, 2025 (JP) 2025-011426
Jul. 4, 2025 (JP) 2025-114041

(51) Int. Cl. G16H 15/00; G10L 15/26; G16H 10/60; G16H 30/40; G16H 50/20; G16H 80/00

(52) U.S. Cl. CPC G16H 15/00; G10L 15/26; G16H 10/60; G16H 30/40; G16H 50/20; G16H 80/00

(57) ABSTRACT

A medical information generation device for generating a document including medical information from voice data of a conversation between a doctor and a patient, the medical information generation device includes a processor configured to: receive the voice data; generate a transcribed text from the received voice data; receive at least the transcribed text and summarize a content of the received text; and analyze a possibility of a disease from image data of an examination of the patient. The processor includes a generation AI model configured to output medical information obtained by incorporating an analysis result derived from analyzing the image data related to the examination of the patient into the summarized text.

### CROSS-REFERENCE TO RELATED APPLICATIONS

This application is based on and claims priority under 35 USC 119 from Japanese Patent Application No. 2025-011426 filed on Jan. 27, 2025 and Japanese Patent Application No. 2025-114041 filed on Jul. 4, 2025, the entire contents of each are incorporated herein by reference.

### BACKGROUND

### 1. Field

The present disclosure relates to a text summarization technique using a generation AI tool, particularly to generating medical information and creating medical documents.

### 2. Description of Related Art

In recent years, with promotion of digital transformation in medical sites, electronic medical record systems have become widespread. Management of medical records and diagnostic information in the related art has mainly involved manual input, which leads to issues with efficiency. For example, a doctor needs to accurately reflect medical care contents in an electronic medical record based on a conversation with a patient.

Further, with the progress of AI technology in image diagnosis, a technique for analyzing image data of an examination received by a patient and analyzing presence or absence of a disease and a possibility thereof with high accuracy has attracted attention. Accordingly, improvement in accuracy of a diagnosis result and earlier detection are expected. Then, means for quickly and effectively reflecting an analysis result in the medical sites is required.

Meanwhile, with evolution of large language models (LLM), a technique for extracting and summarizing necessary information from a huge amount of data has been put into practical use. By utilizing this technique, simple and accurate medical information can be generated.

An electronic medical record system of Patent Literature 1 (JP7441391B) corrects a drug prescription order of an electronic medical record by a large-scale language model. Specifically, a drug prescription is described in a drug prescription document of the electronic medical record, and prescription contents are transcribed to a prompt of the LLM. The LLM makes an answer and a correction while referring to contents of an interview form and the like. When the doctor determines that the correction contents are appropriate, the contents are transcribed to the drug prescription document of the electronic medical record (see paragraph 0034 and FIG. 6).

### SUMMARY OF INVENTION

The electronic medical record system mainly has functions such as summarization, correction, and transcription of necessary medical records. Generation and management of such medical records are frequently required, but it takes much time and effort to analyze examination results and generate a summary. As a result, a decrease in efficiency in medical care work is regarded as a problem, which may eventually lead to a decrease in a quality of medical care and service to the patient.

The present disclosure has been made in view of such circumstances, and an object thereof is to provide a medical information generation device and a medical information generation method that efficiently reduce the workload on a creator by efficiently generating medical information and a medical documents.

A medical information generation device for generating a document including medical information from voice data of a conversation between a doctor and a patient, the medical information generation device includes a processor configured to: receive the voice data; generate a transcribed text from the received voice data; receive at least the transcribed text and summarize a content of the received text; and analyze a possibility of a disease from image data of an examination of the patient, in which the processor includes a generation AI model configured to output medical information obtained by incorporating an analysis result derived from analyzing the image data related to the examination of the patient into the summarized text.

According to the medical information generation device of the present disclosure reduces the workload on a creator by efficiently creating the medical information and the medical document.

### BRIEF DESCRIPTION OF DRAWINGS

FIG. 1 is a schematic diagram of a medical information generation device according to the present disclosure.

FIG. 2 is a diagram illustrating input and output of the medical information generation device.

FIG. 3 is a diagram illustrating an internal configuration of the medical information generation device.

FIG. 4 is a diagram illustrating a flowchart of a medical information generation process.

FIG. 5 is a diagram illustrating a method of analyzing image data.

FIG. 6 is a diagram illustrating a flowchart of summarization processing in the medical information generation process.

FIG. 7 is a diagram illustrating a flowchart of referral letter generation processing in the medical information generation process.

FIG. 8 is a diagram showing a screen example of a referral letter generation application.

FIG. 9 is a diagram showing an example of a generated referral letter.

### DESCRIPTION OF EMBODIMENTS

Hereinafter, an example of the present disclosure will be described with reference to the drawings, but the scope of the present disclosure is not limited to illustrative embodiments described here, and various modifications can be made without departing from the scope of the present disclosure.

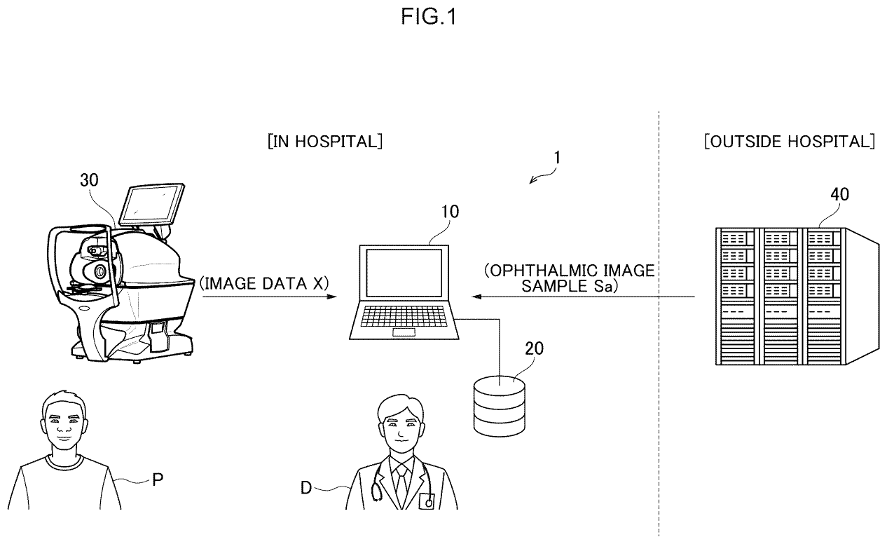

FIG. 1 is an overall diagram of a medical system 1 including a medical information generation device 10 according to an illustrative embodiment of the present disclosure.

The medical system 1 includes the medical information generation device 10 mainly installed in an examination room or the like in a hospital, a trained model 20, an ophthalmic device 30, and a server device 40 mainly installed outside the hospital. The ophthalmic device 30 is connected to the medical information generation device 10 via a network. The ophthalmic device 30 is mainly a fundus camera or an optical coherence tomography (OCT) device, but may include devices such as a slit lamp microscope or a scanning laser ophthalmoscope (SLO).

The medical information generation device 10 is a terminal device such as a desktop PC, a notebook PC, or a tablet computer owned by a medical institution such as a hospital. Hereinafter, a case where a patient P undergoes an examination for glaucoma in the hospital will be described. When the patient P undergoes intraocular pressure measurement, for example, an intraocular pressure value is transmitted to the medical information generation device 10 as numerical data. When a fundus image, an OCT image, or the like of the patient P is captured using the ophthalmic device 30, the image is transmitted to the medical information generation device 10 as image data X. The numerical data such as the intraocular pressure value and the image data X such as the fundus image are stored in the medical information generation device 10 and can be checked by a doctor D.

The server device 40 is connected to the medical information generation device 10 via a network. Therefore, the doctor D can constantly access an ophthalmic image sample Sa (for example, a fundus image including a symptom of glaucoma) which is a result of a past image diagnosis stored in the server device 40. The doctor D can check recorded matters and the like in the electronic medical record, in addition to the result of the ophthalmic diagnosis acquired in another medical institution.

The doctor D determines a possibility of glaucoma in the patient P based on at least the numerical data and the image data X. Further, the doctor D can receive assistance in detecting a lesion or a disease from the medical information generation device 10 and the trained model 20.

The medical information generation device 10 determines the possibility of glaucoma by comparing the image data X of the patient P with a plurality of ophthalmic image samples Sa using the trained model 20. The glaucoma can be generally estimated based on the intraocular pressure value, but glaucoma with normal intraocular pressure also exists. The medical system 1 according to the present disclosure can make a quick and accurate determination without overlooking various symptoms of the glaucoma with the assistance of the trained model 20.

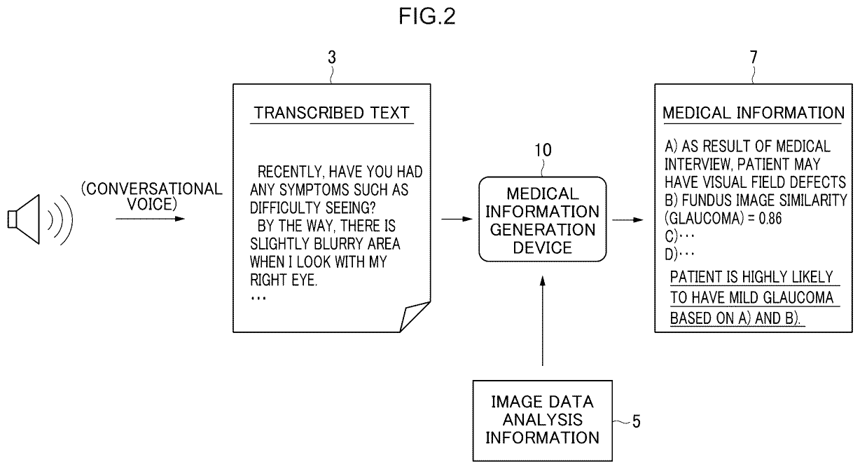

FIG. 2 is a diagram illustrating input and output of the medical information generation device 10. The medical information generation device 10 generates medical information 7 such as an electronic medical record and a referral letter (a formal name is a patient referral document), by summarizing input texts using a so-called generation AI tool.

One piece of the data to be input to the medical information generation device 10 is a transcribed text 3 obtained by converting conversational voice of the doctor and the patient into text. The transcribed text 3 contains, for example, contents of a speech as it is, such as “Recently, have you had any symptoms such as difficulty seeing?” from the doctor.

Another piece of data to be input to the medical information generation device 10 is image data analysis information 5. The image data analysis information 5 is information on a disease that is mainly known from the image data X of the patient. When receiving the transcribed text 3 and the image data analysis information 5, the medical information generation device 10 automatically executes a prompt to generate the medical information 7 in a form including these contents.

As a result of the text generation in the medical information generation device 10, the medical information 7 is output. The medical information 7 is useful information obtained by adding the result of the image data analysis information 5 to the transcribed text 3, and can be described in the electronic medical record. In particular, regarding the image data analysis information 5, it takes time for the doctor D to indicate his/her opinion and convert the opinion into text. In this regard, the medical information generation device 10 can efficiently incorporate the result of the image data analysis information 5 into the medical information 7. The medical information generation device 10 can generate a plurality of patterns of referral letters including the medical information 7.

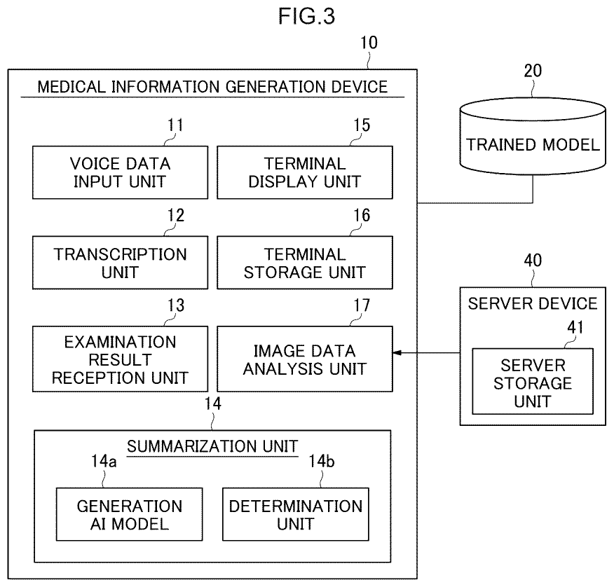

FIG. 3 is a diagram illustrating an internal configuration of the medical information generation device 10 of the present disclosure.

The medical information device 10 includes therein a voice data input unit 11, a transcription unit 12, an examination result reception unit 13, a summarization unit 14, a terminal display unit 15, a terminal storage unit 16, and an image data analysis unit 17. Each of the transcription unit 12, the examination result reception unit 13, the summarization unit 14, and the image data analysis unit 17 is configured using, for example, a processor such as a CPU (Central Processing Unit). By cooperating with a memory (not shown) provided in the medical information generation device 10, the processor enables the processing performed by each of the transcription unit 12, the examination result reception unit 13, the summarization unit 14, and the image data analysis unit 17.

The voice data input unit 11 has a configuration of a microphone (and a recorder) that inputs real-time conversational voice of the doctor D and the patient P during medical interview and medical examination. By automatically recording the conversational voice, it is possible to secure a time during which the doctor D and the patient P directly face each other during medical care, and to provide a better examination environment.

The transcription unit 12 performs transcription on the conversational voice received from the voice data input unit 11 to generate the transcribed text 3 (see FIG. 2). The transcription unit 12 may generate characters including an instruction word, an onomatopoeic word, and the like in the voice data. The transcription unit 12 can also perform transcription on conversational voice recorded in advance in a recording medium such as a voice recorder.

The examination result reception unit 13 receives the image data X transmitted from the ophthalmic device 30. The image data X is an examination image of the patient P that can be captured by the ophthalmic device 30, such as an anterior segment image, a fundus image, or an OCT image (including an anterior segment OCT).

The summarization unit 14 includes a generation AI model 14a and a determination unit 14b. The generation AI model 14a is an existing generation AI tool that adopts a large language model, and generates the medical information 7 by summarizing contents of the transcribed text 3 and the image data analysis information 5. The determination unit 14b determines whether to generate a referral letter as the medical information 7, and to select an appropriate template from a plurality of templates stored in the terminal storage unit 16 during the generation.

The terminal display unit 15 is a display that displays a result of the examination or image diagnosis. The doctor D can check the ophthalmic diagnosis result and the recorded items in the electronic medical record via the terminal display unit 15. A type of the display may be any type such as liquid crystal, plasma, or organic EL, and may be a touch panel type.

The terminal storage unit 16 is a storage medium, such as a semiconductor memory, an optical disk, or a magnetic disk into which data can be written. The ophthalmic image sample Sa in the server device 40 may be able to be downloaded by the doctor D, stored in the terminal storage unit 16, and accessed when necessary. In addition, as patterns of the template for generating the referral letter, at least three types including a reply referral letter, an examination request referral letter, and a treatment request referral letter are stored.

The image data analysis unit 17 analyzes the image data X while referring to the ophthalmic image sample Sa, and determines presence or absence of a disease and a degree of progress (stage). At this time, the image data analysis unit 17 uses the trained model 20. The image data analysis unit 17 is a processor (CPU, GPU, FPGA, or the like) that can mainly perform image analysis on the image data X by AI.

The trained model 20 is a machine learning database in which machine learning is performed to determine a possibility of an eye disease by receiving a relationship between a plurality of ophthalmic image samples Sa and the eye disease as learning data. For example, the machine learning is performed by receiving many fundus images as learning data, and adding results of glaucoma, age-related macular degeneration, and the like to obtain training data. Accordingly, it is possible to output information such as a current degree of progress of the glaucoma and a future incidence rate.

The ophthalmic image samples S may be converted into data groups, each reflecting a criterion of the medical institution to be used or a determination criterion of the doctor in charge. Further, a user may be able to refer to or use an ophthalmic image sample created by specialists in each diseases, including glaucoma and age-related macular degeneration. Accordingly, an image data analysis processing is accurately performed according to a decision of the medical institution or the doctor in charge.

A method for the machine learning is not particularly limited, and various methods such as unsupervised learning and deep learning can be adopted. The trained model 20 may be configured to be connected to the server device 40.

Next, the server device 40 includes a server storage unit 41 therein. The server storage unit 41 is a storage medium, such as a semiconductor memory, an optical disk, or a magnetic disk into which data can be written. The server storage unit 41 stores previously obtained ophthalmic diagnosis results (numerical values of the examination and the ophthalmic image samples Sa). The ophthalmic image samples Sa in the server storage unit 41 are separated into image groups based on eye diseases such as glaucoma and age-related macular degeneration, and also into image groups based on a degree of progress of each eye disease.

A type of the server device 40 is not particularly limited, and may be, for example, a cloud server. In the present illustrative embodiment, the server device 40 is assumed to be of a cloud type and is outside the hospital, but may be a related-art server installed in the hospital.

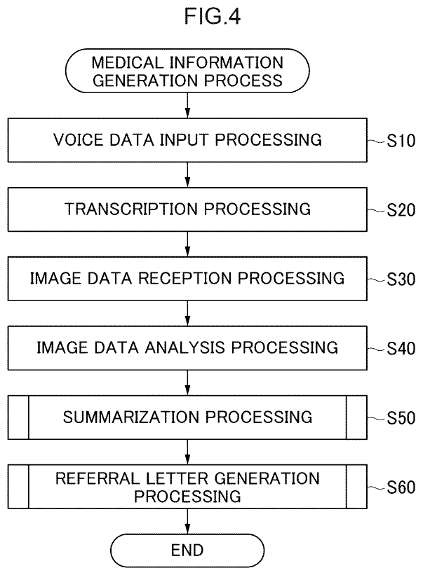

Next, a medical information generation process will be described in detail with reference to FIGS. 4 to 7. The medical information generation process is a process to be performed by the medical information generation device 10, and is premised on a fact that the ophthalmic diagnosis of the patient P is performed.

FIG. 4 shows a flowchart of the medical information generation process. First, the medical information generation device 10 (voice data input unit 11) performs voice data input processing (step S10). Specifically, the medical information generation device 10 inputs conversational voice (voice data) between the doctor D and the patient P during an interview. Here, the voice data may be real-time voice or voice stored in advance in a storage medium. Thereafter, the processing of the medical information generation device 10 proceeds to step S20.

Next, the medical information generation device 10 (transcription unit 12) performs transcription processing on the voice data received in step S10 (step S20). Accordingly, the transcribed text 3 (see FIG. 2) is generated. Thereafter, the processing of the medical information generation device 10 proceeds to step S30.

Next, the medical information generation device 10 (examination result reception unit 13) performs image data reception processing and receives current (or latest) image data (step S30). Here, the image data is a fundus image, an OCT image, or the like (image data X in FIG. 1) acquired by the ophthalmic device 30. Thereafter, the processing of the medical information generation device 10 proceeds to step S40.

Next, the medical information generation device 10 (image data analysis unit 17) performs image data analysis processing (step S40). Specifically, an image group having a high similarity (similarity score) is found by comparing the current image data X with reference image data of each ophthalmic image sample Sa based on image analysis using AI. Since the similarity is a numerical value that contributes to detection of a specific disease, the disease can be estimated with high reliability.

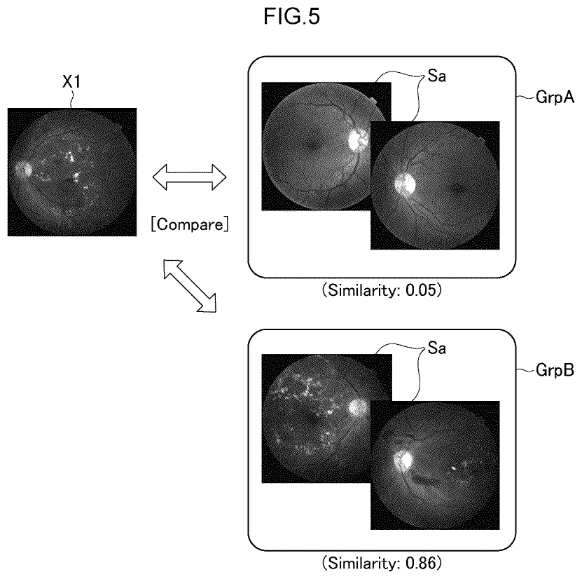

Here, a method of analyzing the image data in this step will be described with reference to FIG. 5. The following analysis is internal processing of the image data analysis unit 17, and is not displayed on the terminal display unit 15 or the like.

Image data X1 in the figure is one piece of the image data X, and is the fundus image of the patient P obtained by the ophthalmic diagnosis. The image data analysis unit 17 extracts a feature from the image data X1. Accordingly, the image data analysis unit 17 extracts, from the image data X1, a feature specific to the presence or absence of an eye disease such as glaucoma, age-related macular degeneration, and diabetic retinopathy, or to a stage of each of the eye diseases.

GrpA is an image group in which the ophthalmic image samples Sa of glaucoma stage 1 are collected, and a feature specific to stage 1 is already extracted. Further, GrpB is an image group in which the ophthalmic image samples Sa of glaucoma stage 2 are collected, and a feature specific to stage 2 is already extracted. In addition, many image groups such as groups of glaucoma stages 3 and 4, groups of age-related macular degeneration stages 1 to 4, and groups of diabetic retinopathy stages 1 to 4 are prepared.

The image data analysis unit 17 sequentially compares the feature of the image data X1 with the feature of each image group, and determines an image group having a highest similarity. In an example of FIG. 5, a similarity between the image data X1 and GrpA is 0.05, whereas a similarity between the image data X1 and GrpB is a high numerical value of 0.86. Therefore, the image data analysis unit 17 determines that the image data X1 is close to the feature of glaucoma stage 2 in GrpB and a possibility is high.

Returning to FIG. 4, the medical information generation device 10 (summarization unit 14) performs summarization processing for generating medical record information in a SOAP format (step S50). The SOAP format refers to a recording format including descriptions of Subjective, Objective, Assessment, and Plan.

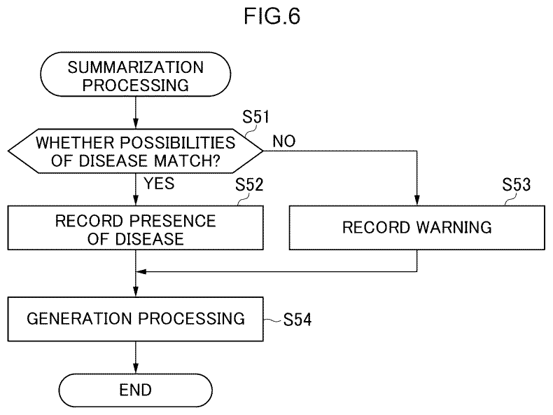

Here, the summarization processing in the medical information generation process (step S50) will be described in detail with reference to FIG. 6. In the processing, the final medical information 7 is generated using the generation AI model 14a of the summarization unit 14.

First, the medical information generation device 10 (determination unit 14b) determines whether possibilities of a disease match (step S51). Here, the medical information generation device 10 determines a final possibility of a disease from a possibility of the disease seen from the medical interview or finding of the doctor derived from the transcribed text 3 and a possibility of the disease seen from the similarity of step S40 derived from the image data analysis information 5. When the possibilities of the disease match, the processing of the medical information generation device 10 proceeds to step S52, and when the possibilities of the disease do not match, the processing of the medical information generation device 10 proceeds to step S53.

When the possibilities of the disease match (“YES” in step S51), the medical information generation device 10 (generation AI model 14a) records information indicating that the patient P is diagnosed with the disease (step S52). Thereafter, the processing of the medical information generation device 10 proceeds to step S54.

On the other hand, when the possibilities of the disease do not match (“NO” in step S51), the medical information generation device 10 records a warning (step S53). This is because there is a possibility that any one of the determinations is incorrect, and is intended to keep the warning without making a determination. Information indicating to recommend re-examination or to point out follow-up may be recorded. Thereafter, the processing of the medical information generation device 10 proceeds to step S54.

Next, the medical information generation device 10 (generation AI model 14a) performs generation processing (step S54). In the processing, processing results of the steps so far are summarized to generate recorded items (medical information 7) for the electronic medical record. Since summarization work is basically the same every time, the medical information generation device 10 may be programmed to automatically execute a prompt for the generation AI tool prepared in advance. Further, the generated recorded items may be automatically written in predetermined columns in the electronic medical record. As described above, the summarization processing ends.

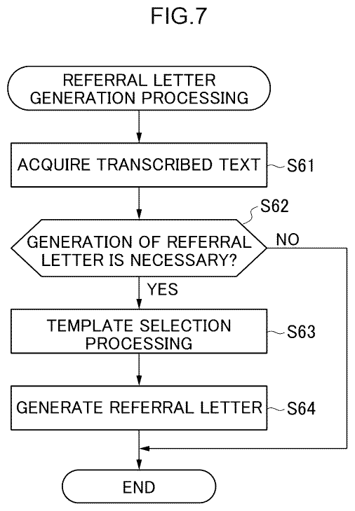

Returning to FIG. 4, the medical information generation device 10 (summarization unit 14) performs referral letter generation processing (step S60). Here, the referral letter generation processing in the medical information generation process will be described in detail with reference to FIG. 7.

First, the medical information generation device 10 acquires the transcribed text (step S61). Specifically, the medical information generation device 10 acquires the transcribed text 3 generated in step S20 of the medical information generation process. The medical information generation device 10 may also acquire the recorded items in a past electronic medical record and the image data analysis information 5. Thereafter, the processing of the medical information generation device 10 proceeds to step S62.

Next, the medical information generation device 10 (determination unit 14b) determines whether it is necessary to generate a referral letter (step S62). The medical information generation device 10 determines the necessity of the referral letter based on the transcribed text 3 and the recorded items in the past electronic medical record. If it is determined that the referral letter generation is necessary, the process of the medical information generation device 10 proceeds to step S63. If it is determined that the referral letter generation is unnecessary, the referral letter generation processing ends without generating additional documents.

When it is determined that the referral letter generation is necessary (“YES” in step S62), the medical information generation device 10 (determination unit 14b) performs template selection processing (step S63). The medical information generation device 10 automatically selects one template that matches a purpose from among the reply referral letter, the examination request referral letter, and the treatment request referral letter.

The selection is not limited to automatic selection, and for example, a mode may be adopted in which the terminal display unit 15 has a referral letter generation button, and one template is selected by an operation of the doctor D. Thereafter, the processing of the medical information generation device 10 proceeds to step S64.

Finally, the medical information generation device 10 (generation AI model 14a) generates the referral letter (step S64). Specifically, the information of the transcribed text 3 and the image data analysis information 5 is summarized using the generation AI tool, and is output in the selected format. Here, the medical information generation device 10 may also be programmed to execute a prompt for the generation AI tool prepared in advance.

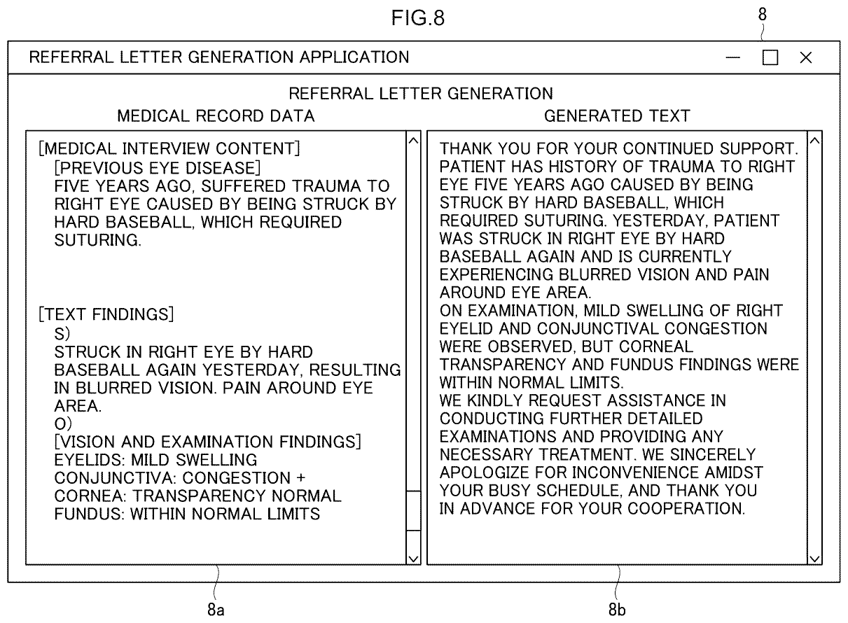

FIG. 8 shows a screen example of a referral letter generation application. The referral letter generation application can be used by the doctor D in the medical information generation device 10, and an application screen 8 is displayed on the terminal display unit 15.

The application screen 8 includes a medical record data frame 8a and a generated sentence frame 8b. In the medical record data frame 8a, the generated recorded items for the electronic medical record are displayed. Records in the electronic medical record include items such as a medical interview content, a character finding (SOAP format), a disease name, an examination treatment, and a prescription.

Further, in the generated sentence frame 8b, a generated sentence for the referral letter based on the recorded items for the electronic medical record is displayed. The doctor D can also add and correct a text in the generated sentence frame 8b. When a manual correction is performed, machine learning can be performed based on contents of the manual correction, which can be used for next and subsequent text generation.

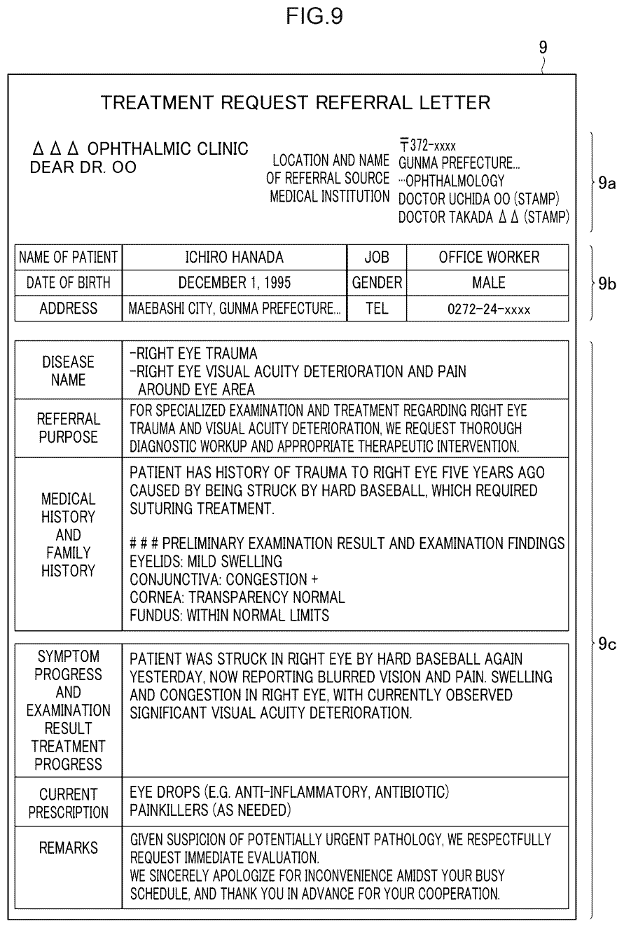

FIG. 9 shows an example of the generated referral letter. Here, an example of a format of the treatment request referral letter will be described.

As shown in the figure, in a treatment request referral letter 9, basic information 9a such as a referral destination and a referral source, personal information 9b of a referring patient, and diagnostic information 9c such as a disease name, a referral purpose, and a symptom progress are described in this order from the top. The personal information 9b is transcribed from the records of the electronic medical record or the like, and generated sentences generated by the referral letter generation application are transcribed as the diagnostic information 9c.

In particular, in the diagnostic information 9c, texts corresponding to the generated sentences of FIG. 8 are automatically written in respective frames of the disease name, the referral purpose, the symptom progress, and the like. Therefore, the doctor D can efficiently generate the referral letter, and a burden is reduced.

The medical information generation device 10 may generate the referral letter in a form including the presence or absence of a specific disease, a warning, and the like among output contents of the summarization processing generated in step S50 of the medical information generation process.

The medical information generation device 10 is not limited to describing information on a disease in the medical document. The medical information generation device 10 can be used to record information on suggestion of appropriate treatment plans, whether a prescribed medicine is appropriate (information on contraindicated drugs), or the like in the medical document. Therefore, the medical information generation device 10 is useful for ensuring safety and efficiency of medical care. As described above, the referral letter generation processing and the medical information generation process end.

The present disclosure is not limited to the illustrative embodiment described above and may be implemented in various modes without departing from the gist thereof. Although the eye disease has been mainly described above as an example, a target of the medical information generation device 10 is not limited to the eye disease. For example, in a case of diseases such as asthma, diabetes, and cancer, the medical information can be generated based on the transcribed text 3 generated from voice data of medical interview and the image data analysis information 5 generated from image data such as X-ray, CT, and MRI.

In the above illustrative embodiment, the medical information generation device 10 generates the medical information 7 mainly from the transcribed text 3 and the image data analysis information 5, but the present invention is not limited thereto. The input data of the examination result of the patient may include numerical data such as visual acuity, eye refractive power, and intraocular pressure value in a case of ophthalmology, and numerical data such as body temperature, blood pressure, and blood glucose level in other fields. The medical information generation device 10 may generate the medical information 7 to include the numerical data.

The generated medical information 7 may be feedback data for the generation AI model 14a to generate new medical information 7. For example, the treatment request referral letter 9 (see FIG. 9) is stored in the terminal storage unit 16 or the server storage unit 41. When generating the new medical information 7, the generation AI model 14a may refer to a previous treatment request referral letter 9, try to generate a summary or a text several times if necessary, and output a final version.

Further, in an ophthalmic examination, both hands of a doctor may be occupied occasionally. In such a case, voice data input of the medical information 7 may be utilized. For example, there is an examination according to an applanation tonometer method in which the intraocular pressure is measured while observing an eyeball of the patient using a slit lamp microscope. In this case, if the medical information generation device 10 is used, a measurement value or a symptom can be directly input to the electronic medical record by voice of the doctor.

A result of a visual acuity examination performed by a doctor or a nurse may be input to the electronic medical record by voice. The transcribed text 3 generated from the voice includes a measurement value of the examination result. Regarding the measurement value, for example, the summarization unit 14 extracts only numerical values based on units (mmHg) of the measurement values and a suggestion made during a speech (words such as “an intraocular pressure of a left eye” and “a visual acuity of a right eye”), and the numerical values are constructed and automatically input in a predetermined column of the electronic medical record (as secondarily usable data).

If the voice data can be input, the doctor or nurse does not need to input data, which leads to a reduction in overall examination and examination time. In this aspect, processing such as image data analysis is unnecessary.

### Appendix

At least the following contents are disclosed by the above description. The medical system 1 of the present disclosure has the following operations and effects.

(1) A medical information generation device for generating a document including medical information from voice data of a conversation between a doctor and a patient, the medical information generation device including a processor configured to:
receive the voice data;
generate a transcribed text from the received voice data;
receive at least the transcribed text and summarize a content of the received text; and
analyze a possibility of a disease from image data of an examination of the patient, in which
the processor includes a generation AI model configured to output medical information obtained by incorporating an analysis result derived from analyzing the image data related to the examination of the patient into the summarized text.

In the medical information generation device of the present disclosure, the processor receives the voice data of the conversation between the doctor and the patient, and performs transcription of the voice data to generate the transcribed text. The processor analyzes a possibility of a disease using the image data of the examination performed on the patient this time.

The processor (generation AI model) efficiently generates a medical document by outputting the medical information obtained by incorporating the analysis result of the image data to the text summarized based on the voice data. Thus, the present device can reduce s burden on an operator.

(2) The medical information generation device according to (1), further including:
a plurality of image groups each including ophthalmic image samples classified by disease progress or disease type, in which
in the analyzing of the image data, the processor analyzes a possibility of a disease contained in the image data from a similarity between a feature of the image data and a feature of each of the ophthalmic image samples.

In the analyzing of the image data, the processor compares the image data of the patient of this time with each of the ophthalmic image samples (classified by the disease progress or the disease type), and analyzes whether the image data of the patient has a lesion or a sign of disease from the similarity. The present device can reflect, as the medical information, an analysis result obtained by analyzing the image data of the patient.

(3) The medical information generation device according to (1) or (2), in which
in a case where a disease estimated from the summarized text matches a disease estimated from the analysis result, the processor records the medical information as the patient's disease.

When the disease estimated from the summarized text matches the disease estimated from the result of the image data, there is a high possibility that the patient has the disease. Therefore, the processor records the medical information including items related to the disease. Accordingly, the present device can enhance contents of the medical information.

(4) The medical information generation device according to (1) or (2), further including:
in a case where the medical information is used for a referral letter, a plurality of templates formatted as referral letters are provided, in which
the generation AI model records the medical information to match the selected template.

Since the referral letter may be handled as the medical information, the plurality of templates are prepared in advance. Since the generation AI model records the medical information to match the selected template, a referral letter in a predetermined format can be easily generated.

(5) A medical information generation method including:
a voice data input step of receiving voice data from a conversation between a doctor and a patient;
a transcription step of generating a transcribed text from the received voice data;
a summarization step of receiving at least the transcribed text and summarizing a content of the received text; and
an image data analysis step of analyzing a possibility of a disease using image data of an examination of the patient, in which
the summarization step includes a generation step of outputting medical information obtained by incorporating an analysis result from the image data analysis step into the summarized text.

In the medical information generation method of the present disclosure, the voice data of the conversation between the doctor and the patient is input by the voice data input step, and the transcription of the voice data is performed by the transcription step to generate the transcribed text. In the image data analysis step, a possibility of a disease is analyzed using the image data of the examination performed on the patient this time.

In the summarization step (generation step), the medical information obtained by adding the analysis result of the image data to the text summarized based on the voice data is output. Accordingly, by the present method, the medical document can be efficiently generated, and the workload of the operator can be reduced.

(6) The medical information generation method according to (5), in which
a plurality of image groups each including ophthalmic image samples classified based on disease progress or disease type are provided, and
in the image data analysis step, a possibility of a disease contained in the image data is analyzed from a similarity between a feature of the image data and a feature of each of the ophthalmic image samples.

In the image data analysis step, the image data of the patient of this time is compared with each of the ophthalmic image samples (classified by the disease progress or the disease type), and whether the image data of the patient has a lesion or a sign of disease is analyzed from the similarity. By the present method, an analysis result obtained by analyzing the image data of the patient can be reflected as the medical information.

(7) The medical information generation method according to (5) or (6), in which
in the summarization step, in a case where a disease estimated from the summarized text matches a disease estimated from the analysis result, the medical information is recorded as the patient's disease.

When the disease estimated from the summarized text matches the disease estimated from the analysis result of the image data, there is a high possibility that the patient has the disease. Therefore, in the summarization step, the medical information including the items related to the disease is recorded. Accordingly, by the present method, the contents of the medical information can be enhanced.

(8) The medical information generation method according to (5) or (6), in which
in a case where the medical information is used for a referral letter, a plurality of templates formatted as the referral letters to be output are provided, and
in the generation step, the medical information is recorded to match the selected template.

Since the referral letter may be handled as the medical information, the plurality of templates are prepared in advance. Since the medical information is recorded to match the selected template in the generation step, a referral letter in a predetermined format can be easily generated.

(9) The medical information generation method according to (5) or (6), in which
in the summarization step, a prompt prepared in advance is provided to a generation AI model to generate the medical information.

Since the method of finally generating the medical information is basically the same every time, in the summarization step, the prompt prepared in advance is given to the generation AI model to generate the medical information, for example, at a time point when input information is prepared. Accordingly, the medical information can be quickly generated by the present method.

(10) A medical information generation device for generating a document including medical information from voice data, the medical information generation device including a processor configured to:
receive the voice data;
generate a transcribed text from the received voice data; and
receive at least the transcribed text and summarize a content of the received text, in which
the generated transcribed text is automatically entered into a predetermined column of an electronic medical record.

In the medical information generation device of the present disclosure, the processor receives the voice data including a symptom, measurement values, and the like estimated by an examination, and performs transcription of the voice data to generate the transcribed text. The transcribed text is structured and automatically entered into a predetermined column of the electronic medical record. The processor may extract only measurement values, such as visual acuity and intraocular pressure, and the like from the transcribed text and input them into the predetermined column.

While illustrative illustrative embodiments have been described above with reference to the accompanying drawings, the present disclosure is not limited to such examples. It will be apparent to those skilled in the art that various modifications, alterations, substitutions, additions, deletions, and equivalents can be conceived within the scope of the claims, and they are also considered to fall within the technical scope of the present disclosure. Furthermore, within the scope without departing from the spirit of the invention, the respective components in the above-described illustrative embodiments may be combined in any combination as appropriate.

What is claimed is:

1. A medical information generation device for generating a document including medical information from voice data of a conversation between a doctor and a patient, the medical information generation device comprising a processor configured to:
receive the voice data;
generate a transcribed text from the received voice data;
receive at least the transcribed text and summarize a content of the received text; and
analyze a possibility of a disease from image data of an examination of the patient,
wherein the processor includes a generation AI model configured to output medical information obtained by incorporating an analysis result derived from analyzing the image data related to the examination of the patient into the summarized text.

2. The medical information generation device according to claim 1, further comprising:
a plurality of image groups each including ophthalmic image samples, classified by disease progression or disease type,
wherein in the analyzing the image data, the processor analyzes a possibility of a disease contained in the image data from a similarity between a feature of the image data and a feature of each of the ophthalmic image samples.

3. The medical information generation device according to claim 1, wherein in a case where a disease estimated from the summarized text matches a disease estimated from the analysis result, the processor records the medical information as the patient's disease.

4. The medical information generation device according to claim 1, further comprising:
in a case where the medical information is used for a referral letter, a plurality of templates formatted as referral letters are provided,
wherein the generation AI model records the medical information to match the selected template.

5. A medical information generation method comprising:
a voice data input step of receiving voice data from a conversation between a doctor and a patient;
a transcription step of generating a transcribed text from the received voice data;
a summarization step of receiving at least the transcribed text and summarizing a content of the received text; and
an image data analysis step of analyzing a possibility of a disease from image data of an examination of the patient,
wherein the summarization step includes a generation step of outputting medical information obtained by incorporating an analysis result from the image data analysis step into the summarized text.

6. The medical information generation method according to claim 5,
wherein a plurality of image groups, each including ophthalmic image samples classified based on disease progression or disease type, are provided, and
wherein in the image data analysis step, a possibility of a disease contained in the image data is analyzed from a similarity between a feature of the image data and a feature of each of the ophthalmic image samples.

7. The medical information generation method according to claim 5, wherein in the summarization step, in a case where a disease estimated from the summarized text matches a disease estimated from the analysis result, the medical information is recorded as the patient's disease.

8. The medical information generation method according to claim 5,
wherein in a case where the medical information is used for a referral letter, a plurality of templates formatted as the referral letters to be output are provided, and
wherein in the generation step, the medical information is recorded to match the selected template.

9. The medical information generation method according to claim 5, wherein in the summarization step, a prompt prepared in advance is provided to a generation AI model to generate the medical information.
